(19) United States
(12) Patent Application Publication
(10) Pub. No.: US 2023/0052964 A1
(43) Pub. Date: Feb. 16, 2023

(54) MANUAL SUCTION DEVICE FOR THROMBUS CAPTURE AND REPERFUSION

(71) Applicant: Boston Scientific Medical Device Limited, Galway (IE)

(72) Inventors: Rajivkumar Singh, Thane (IN); Mihir Sukhatme, Thane (IN); Deepak Kumar Sharma, Muzaffarnagar (IN)

(73) Assignee: Boston Scientific Medical Device Limited, Galway (IE)

(21) Appl. No.: 17/885,003

(22) Filed: Aug. 10, 2022

Related U.S. Application Data
(60) Provisional application No. 63/231,919, filed on Aug. 11, 2021.

(51) Int. Cl. A61M 39/22; A61M 1/36

(52) U.S. Cl. CPC A61M 39/22; A61M 1/3627

(57) ABSTRACT

A medical device is adapted for use in assisting with mechanical thrombectomy. The medical device may include a housing that is adapted to be held within a user's hand and a pump that is secured within the housing. A fluid inlet is adapted to receive fluid that is pulled towards the pump and is fluidly coupled with the pump. A fluid outlet is adapted to expel fluid away from the pump and is fluidly coupled with the pump. As the pump is operated by the user, fluid is alternatively pulled into the fluid inlet and expelled through the fluid outlet. In some cases, the fluid may be blood or other bodily fluids.

### CROSS-REFERENCE TO RELATED APPLICATIONS

This application claims the benefit of priority under 35 U.S.C. § 119 of U.S. Provisional Application No. 63/231,919, filed Aug. 11, 2021, the entire disclosure of which is hereby incorporated by reference.

### TECHNICAL FIELD

The disclosure is directed to suction devices and more particularly to manual suction devices that can be used for capturing thrombi from a patient's vasculature while returning blood to the patient.

### BACKGROUND

There are a variety of medical procedures and medical conditions that may result in unwanted material within a patient's bloodstream. A patient may have thrombolytic material within their bloodstream. There are medical devices intended for removal of thrombolytic material. Of the known medical devices and methods, each has certain advantages and disadvantages. There is an ongoing need to provide alternative medical devices as well as alternative methods for manufacturing and using medical devices.

### SUMMARY

The disclosure is directed to several alternative designs, materials and methods of manually operated mechanical suction devices. An example of the disclosure may be found in a medical device that includes a housing and a pump secured within the housing. A fluid inlet is fluidly coupled with the pump and is adapted to receive fluid pulled towards the pump. A fluid outlet is fluidly coupled with the pump and is adapted to expel fluid away from the pump. A fluid path extends from the fluid inlet to the fluid outlet. As the pump is operated by the user, fluid is alternatively pulled into the fluid inlet and expelled through the fluid outlet.

Alternatively or additionally, the pump may include a manually operated pump.

Alternatively or additionally, the pump may include a cylinder formed within the housing, a piston slidingly disposed within the cylinder, a connecting rod pivotably secured to the piston and a rotating disk having a handle to rotate the disk, the connecting rod pivotably secured to the rotating disk at a position offset from a center of the rotating disk such that when the rotating disk is rotated, the connecting rod causes the piston to translate back and forth within the cylinder.

Alternatively or additionally, the pump may further include a handle adapted to be coupled with the rotating disk such that rotating the handle causes the rotating disk to rotate.

Alternatively or additionally, the handle may be adapted to be releasably coupled with the rotating disk.

Alternatively or additionally, the handle may be adapted to form an interference fit with the rotating disk.

Alternatively or additionally, the medical device may further include a one way inlet valve disposed within the fluid inlet such that fluid is allowed to flow into the fluid inlet when the piston is moved away from the fluid path but is prevented from flowing out of the fluid inlet when the piston is moved towards the fluid path and a one way outlet valve disposed within the fluid outlet such that fluid is allowed to flow out of the fluid outlet when the piston is moved towards the fluid path but is prevented from flowing into the fluid outlet when the piston is moved away from the fluid path.

Another example of the disclosure may be found in a medical device that is adapted to facilitate mechanical thrombectomy. The medical device includes a housing adapted to be held within a user's hand and a pump secured within the housing, the pump including a handle that is adapted to be engaged by a user's other hand. A fluid inlet is fluidly coupled with the pump and is adapted to accommodate a first fluid line bringing fluid to the fluid inlet. A fluid outlet is fluidly coupled with the pump and is adapted to accommodate a second fluid line taking fluid away from the fluid outlet. As the pump is operated by the user, fluid is alternatively pulled into the fluid inlet and expelled through the fluid outlet.

Alternatively or additionally, the pump may include a piston slidingly disposed relative to the housing, a connecting rod pivotably secured to the piston and a rotating disk having a handle to rotate the disk, the connecting rod pivotably secured to the rotating disk at a position offset from a center of the rotating disk such that when the rotating disk is rotated, the connecting rod causes the piston to translate back and forth relative to the housing.

Alternatively or additionally, the medical device may further include a handle that is adapted to be releasably coupled with the rotating disk.

Alternatively or additionally, the medical device may further include a handle that handle is adapted to form an interference fit with the rotating disk.

Alternatively or additionally, the medical device may further include a one way inlet valve that is disposed within the fluid inlet and is adapted to only allow fluid to flow into the fluid inlet and a one way outlet valve that is disposed within the fluid outlet and is adapted to only allow fluid to flow out of the fluid outlet.

Alternatively or additionally, the piston may include a rubber feature that seals against an interior of the housing.

Another example of the disclosure may be found in a system for capturing unwanted materials within blood flowing through a patient's vasculature. The system includes a blood filter adapted to capture the unwanted materials and a medical device that is adapted to facilitate mechanical thrombectomy. The medical device includes a housing adapted to be held within a user's hand and a pump secured within the housing, the pump including a handle that is adapted to be engaged by a user's other hand. A fluid inlet is fluidly coupled with the pump and is adapted to accommodate a first fluid line bringing fluid to the fluid inlet. A fluid outlet is fluidly coupled with the pump and is adapted to accommodate a second fluid line taking fluid away from the fluid outlet. As the pump is operated by the user, fluid is alternatively pulled into the fluid inlet and expelled through the fluid outlet. The blood filter is adapted to be fluidly coupled with the fluid inlet and blood exiting the fluid outlet is directed back to the patient.

Alternatively or additionally, the blood exiting the fluid outlet may be returned directly to the patient.

Alternatively or additionally, the blood exiting the fluid outlet may be captured and stored for subsequent return to the patient.

Alternatively or additionally, the unwanted materials may include embolic material.

Alternatively or additionally, the unwanted materials may include foreign matter not normally part of the patient's blood.

Another example of the disclosure may be found in a medical device adapted for removing unwanted materials within blood flowing through a patient's vasculature. The medical device includes a housing that is adapted to be held within a user's hand and a pump that is secured within the housing. The pump includes a cylinder formed within the housing, a piston slidingly disposed within the cylinder, a connecting rod pivotably secured to the piston and a rotating disk having a handle to rotate the disk, the connecting rod pivotably secured to the rotating disk at a position offset from a center of the rotating disk such that when the rotating disk is rotated, the connecting rod causes the piston to translate back and forth within the cylinder. A fluid inlet is fluidly coupled with the pump and is adapted to receive fluid pulled towards the pump. A fluid outlet is fluidly coupled with the pump and is adapted to expel fluid away from the pump. As the pump is operated by the user, fluid is alternatively pulled into the fluid inlet and expelled through the fluid outlet.

Alternatively or additionally, the medical device may further include a handle that is releasably securable relative to the rotating disk.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE FIGURES

The disclosure may be more completely understood in consideration of the following description of in connection with the accompanying drawings, in which:

FIG. 1 is a perspective view of an illustrative medical device, shown in a user's hand;

FIG. 2 is a plan view of the illustrative medical device of FIG. 1, shown without a handle;

FIG. 3 is a perspective view of the illustrative medical device of FIG. 1, with the handle present but not attached;

FIG. 4 is a plan view of the illustrative medical device of FIG. 1 with one or more cover elements removed to show internal structure; and

FIG. 5 is a schematic view of a mechanical thrombectomy system including the illustrative medical device of FIG. 1.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

Definitions of certain terms are provided below and shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a,” “an,” and “the” include or otherwise refer to singular as well as plural referents, unless the content clearly dictates otherwise. As used in this specification and the appended claims, the term “or” is generally employed to include “and/or,” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

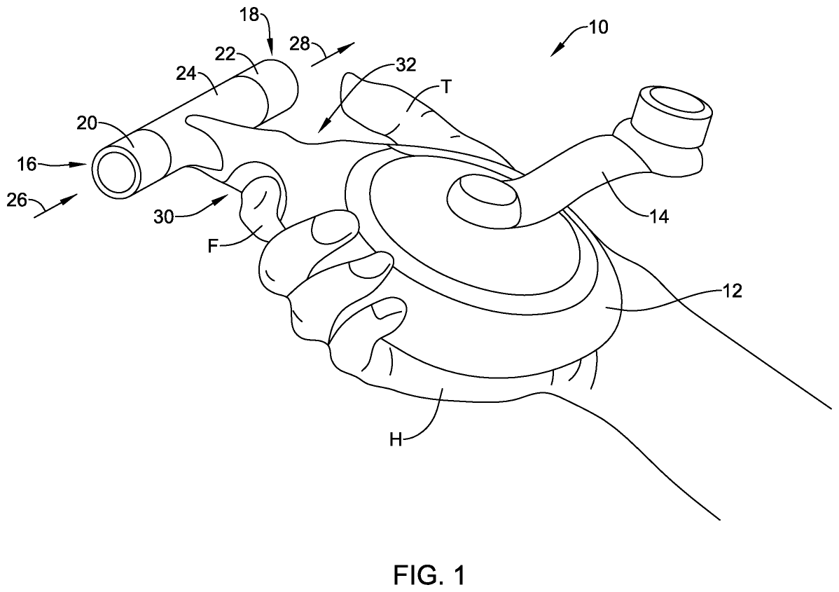

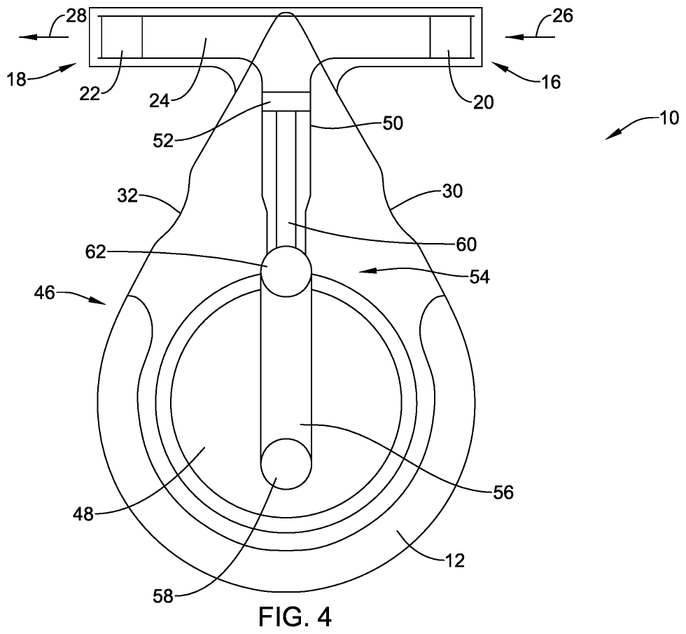

FIG. 1 is a perspective view of an illustrative medical device 10. In some cases, the illustrative medical device 10 may be used as a source of suction in order to pull a fluid such as blood or other bodily fluids into the medical device 10 as well as to pump the fluid such as blood or other bodily fluids out of the medical device 10 for subsequent return to the patient. The overall system will be discussed with respect to FIG. 4. In FIG. 1, the medical device 10 can be seen having a housing 12 that fits into a user's hand H. The medical device 10 may also be used while resting or even being mounted on a table or other surface, for example. The medical device 10 includes a handle 14 that is adapted to permit the user to actuate the handle 14 using their other hand. As illustrated, the user is holding the medical device 10 in their right hand H, meaning that they can actuate the handle 14 with their left hand (not shown). It will be appreciated that the medical device 10 can easily be held in a user's left hand, meaning that the user would then use their right hand H to actuate the handle 14. This may be determined based on whether the user is left-handed or right-handed, for example.

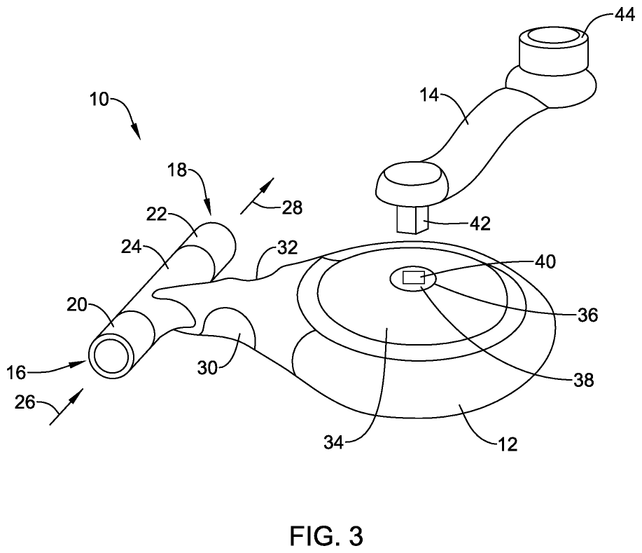

The medical device 10, which may be considered as including or even being a fluid pump, includes a fluid inlet 16 and a fluid outlet 18. In some cases, as shown, the medical device 10 may include a one way inlet valve 20 that is disposed proximate the fluid inlet 16 and a one way outlet valve 22 that is disposed proximate the fluid outlet 18. A fluid path 24 may be considered as extending between the fluid inlet 16 and the fluid outlet 18. As shown, the fluid inlet 16, the fluid outlet 18 and the fluid path 24 extending therebetween is illustrated as being transparent. In some cases, the fluid inlet 16, the fluid outlet 18 and the fluid path 24 extending therebetween may be formed of a transparent or substantially transparent polymeric material in order to allow the user to visualize the flow of fluid through the medical device 10. As will be discussed with respect to FIG. 3, the fluid path 24 also extends down into the medical device 10.

The one way inlet valve 20 may be adapted to permit fluids such as blood or other bodily fluids to flow into the fluid inlet 16 in a direction indicated by an arrow 26 but not allow fluids such as blood or other bodily fluids to flow in an opposite direction, opposing the arrow 26. The one way outlet valve 22 may be adapted to permit fluids such as blood or other bodily fluids to flow out of the fluid outlet 18 in a direction indicated by an arrow 28 but not allow fluids such as blood or other bodily fluids to flow in an opposite direction, opposing the arrow 28.

The housing 12 includes recessed areas 30 and 32 on either side of the housing 12 that facilitate the user easily holding the medical device 12 in their hand H. As can be seen, the recessed area 30 enables the user's finger F to fit into the recessed area 30 while the recessed area 32 enables the user's thumb T to fit into the recessed area 32. It will be appreciated that if the user was holding the medical device 10 in their left hand (not shown), the recessed area 30 would accommodate the user's thumb T while the recessed area 32 would accommodate the user's finger F.

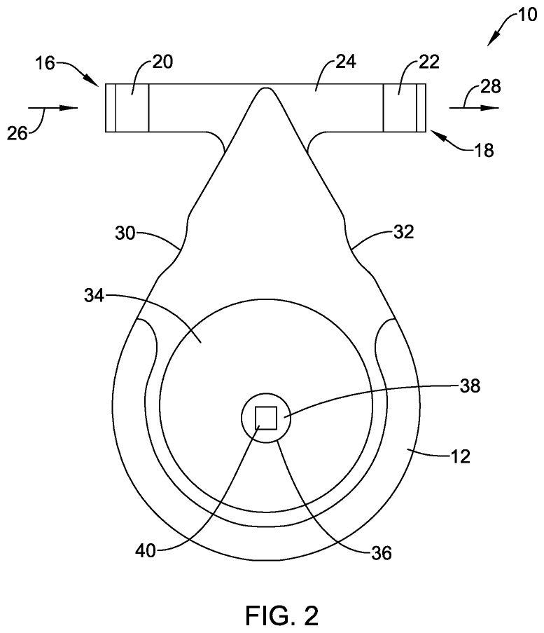

FIG. 2 provides a plan view of the medical device 10. The plan view further illustrates the recessed areas 30 and 32. The handle 14 has been removed in this view. The housing 12 may include a cover 34 that is fixed relative to the housing 12 but may include an aperture 36 that accommodates a drive mechanism 38 that extends through the aperture 36 in the cover 34. The drive mechanism 38 is adapted to rotate relative to the cover 34 when the handle 14 is secured to the drive mechanism 38 and the user rotates the handle 14. In some cases, there may be an interference fit formed between the handle 14 and the drive mechanism 38. As illustrated, the drive mechanism 38 includes a square aperture 40 that is adapted to accommodate a corresponding square peg formed on the handle 14, as will be shown in FIG. 3. While a square aperture 40 is shown, it will be appreciated that the drive mechanism 38 may include an aperture having any desired shape, such as square, rectangular, hexagonal or other polygonal shape, triangular or the like, as long as the handle 14 includes a complementary shaped peg, and as long as the particular shape and the corresponding interaction between the aperture and the peg enables an interference fit. For example, a round aperture and corresponding round peg would not work as well.

FIG. 3 shows that the handle 14 includes a square peg 42 that is adapted to fit snugly into the square aperture 40. As noted, the peg 42 may have any of a variety of different shapes, as long as that shape is complementary to that of the aperture 40 and the interaction therebetween facilitates an interference fit. In some cases, as shown, the handle 14 may include a knob 44 that rotates relative to the handle 14. This facilitates actuating the handle 14 because as the user grasps the knob 44 and thus rotates the handle 14, the knob 44 is able to remain at a fixed position relative to the user's hand as the user actuates the handle 14. It will be appreciated that the particular size and shape of the handle 14 shown is merely illustrative, as any of a variety of different handle shapes could be used. As will be discussed with respect to FIG. 4, the handle 14 may be turned in either direction in order to pump fluids such as blood or other bodily fluids through the medical device 10.

FIG. 4 is a plan view of the medical device 10. If FIG. 3 is considered to be a “front” view of the medical device 10, then FIG. 4 may be considered as being a “back” view, with one or more cover elements removed to show features of a pump mechanism 46. The pump mechanism 46 includes several components, including a rotating disk 48. While not seen in this view, it will be appreciated that the drive mechanism 38 (FIG. 3) is coupled with the rotating disk 48 such that when the drive mechanism 38 is rotated, the rotating disk 48 is caused to rotate in response. While not illustrated, in some cases it is contemplated that an electric motor may instead be used to rotate the rotating disk 48. If an electric motor is used, the medical device would also include an on/off switch or other mechanism to control operation of the electric motor. In some cases, an air driven motor, driven for example by compressed air interacting with a rotor, may alternatively be used.

A cylinder 50 is formed within the housing 12. The cylinder 50 may be a separate piece that is separately formed and then disposed within the housing 12. In some cases, the cylinder 50 may be integrally formed as part of the housing 12. A piston 52, which may include a rubber component such as an O ring in order to form a better seal against a wall of the cylinder 50, is slidingly disposed within the cylinder 50. A connecting rod 54 extends from the rotating disk 48 to the piston 52 in order to convert rotation of the rotating disk 48 into translation back and forth of the piston 52 within the cylinder 50. The connecting rod 54 may be considered as including several components. A first connecting rod portion 56 is pivotably coupled to the rotating disk 48 at a pivot point 58. A second connecting rod portion 60 extends up to the piston 52 and couples to the first connecting rod portion 56 at a pivot point 62. In some cases, the coupling between the second connecting rod portion 60 and the piston 52 may include another pivot mechanism.

As the handle 14 is rotated, causing the rotating disk 48 to rotate, it will be appreciated that the piston 52 translates back and forth (or up and down, in the illustrated orientation) within the cylinder 50. As the piston 52 moves downward, or away from the fluid path 24, a resulting suction causes fluids such as blood or other bodily fluids to enter the fluid inlet 16 in the direction indicated by the arrow 26. The one way outlet valve 22 prevents fluid from flowing in a direction opposite that of the arrow 28. As the piston 52 moves upward, or towards the fluid path 24, a resulting pressure causes fluids such as blood or other bodily fluids to exit the fluid outlet 18 in the direction indicated by the arrow 28. The one way inlet valve 20 prevents fluids such as blood or other bodily fluids from exiting the fluid inlet 16 in a direction opposing that of the arrow 26.

As the handle 14 is rotated, therefore, the medical device 10 will alternately pull fluids such as blood or other bodily fluids into the medical device 10 via the fluid inlet 16 and push fluids such as blood or other bodily fluids out of the medical device 10 via the fluid outlet 18. The medical device 10 is insensitive to orientation. The medical device 10 will work equally well whether held in the illustrated orientation, or upside down, or any other orientation. The medical device 10 can be stopped or started, meaning the user either stops rotating the handle 14 or starts rotating the handle 14, at any handle orientation.

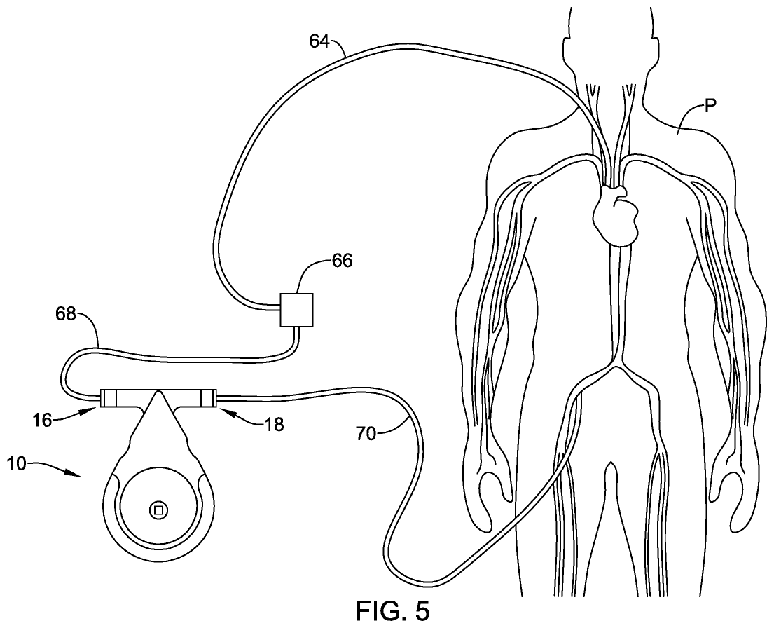

FIG. 5 is a schematic view, showing a possible use for the medical device 10. A patient P is shown. A first fluid line 64 can be seen exiting the patient P. There may be a hemostatic valve disposed between the patient P and the first fluid line 64, for example. The first fluid line 64 extends to a filter assembly 66. As the medical device 10 is actuated, and blood is pulled through the first fluid line 64 into and through the filter assembly 66, the filter assembly 66 may capture any unwanted material within the blood, such as but not limited to thrombolytic material. A second fluid line 68 fluidly couples the filter assembly 66 with the fluid inlet 16 of the medical device 10. A third fluid line 70 fluidly couples the fluid outlet 18 of the medical device 18 with the patient P for reperfusion. The third fluid line 70 may extend to the patient P via a hemostatic valve, for example. In some cases, the third fluid line 70 may instead be coupled with a blood bag or other structure that is adapted to collect the blood before the blood is subsequently returned to the patient P.

It will be appreciated that a variety of different materials may be used in forming the medical device 10. In some cases, a variety of different metals may be used. Illustrative but non-limiting examples of suitable metals include titanium, stainless steel, magnesium, cobalt chromium and others. In some cases, some elements may be made of Nitinol. In some embodiments, for example, the devices described herein may include any suitable polymeric material, including biocompatible materials such as polyurethane or silicone. Suitable polymers include PEEK (polyetheretherketone) and Polycarbonate. Other suitable polymers include but are not limited to polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

What is claimed is:

1. A medical device, comprising:
a housing;
a pump secured within the housing;
a fluid inlet fluidly coupled with the pump, the fluid inlet adapted to receive fluid pulled towards the pump;
a fluid outlet fluidly coupled with the pump, the fluid outlet adapted to expel fluid away from the pump; and
a fluid path extending from the fluid inlet to the fluid outlet;
wherein as the pump is operated by the user, fluid is alternatively pulled into the fluid inlet and expelled through the fluid outlet.

2. The medical device of claim 1, wherein the pump comprises a manually operated pump.

3. The medical device of claim 1, wherein the pump comprises:
a cylinder formed within the housing;
a piston slidingly disposed within the cylinder;
a connecting rod pivotably secured to the piston; and
a rotating disk having a handle to rotate the disk, the connecting rod pivotably secured to the rotating disk at a position offset from a center of the rotating disk such that when the rotating disk is rotated, the connecting rod causes the piston to translate back and forth within the cylinder.

4. The medical device of claim 3, wherein the pump further comprises a handle adapted to be coupled with the rotating disk such that rotating the handle causes the rotating disk to rotate.

5. The medical device of claim 4, wherein the handle is adapted to be releasably coupled with the rotating disk.

6. The medical device of claim 4, wherein the handle is adapted to form an interference fit with the rotating disk.

7. The medical device of claim 1, further comprising:
a one way inlet valve disposed within the fluid inlet such that fluid is allowed to flow into the fluid inlet when the piston is moved away from the fluid path but is prevented from flowing out of the fluid inlet when the piston is moved towards the fluid path; and
a one way outlet valve disposed within the fluid outlet such that fluid is allowed to flow out of the fluid outlet when the piston is moved towards the fluid path but is prevented from flowing into the fluid outlet when the piston is moved away from the fluid path.

8. A medical device adapted to facilitate mechanical thrombectomy, the medical device comprising:
a housing adapted to be held within a user's hand;
a pump secured within the housing, the pump including a handle that is adapted to be engaged by a user's other hand;
a fluid inlet fluidly coupled with the pump and adapted to accommodate a first fluid line bringing fluid to the fluid inlet;
a fluid outlet fluidly coupled with the pump and adapted to accommodate a second fluid line taking fluid away from the fluid outlet;
wherein as the pump is operated by the user, fluid is alternatively pulled into the fluid inlet and expelled through the fluid outlet.

9. The medical device of claim 8, wherein the pump comprises:
a piston slidingly disposed relative to the housing;
a connecting rod pivotably secured to the piston; and
a rotating disk having a handle to rotate the disk, the connecting rod pivotably secured to the rotating disk at a position offset from a center of the rotating disk such that when the rotating disk is rotated, the connecting rod causes the piston to translate back and forth relative to the housing.

10. The medical device of claim 9, further comprising a handle that is adapted to be releasably coupled with the rotating disk.

11. The medical device of claim 9, further comprising a handle that handle is adapted to form an interference fit with the rotating disk.

12. The medical device of claim 8, further comprising:
a one way inlet valve disposed within the fluid inlet and adapted to only allow fluid to flow into the fluid inlet; and
a one way outlet valve disposed within the fluid outlet and adapted to only allow fluid to flow out of the fluid outlet.

13. The medical device of claim 9, wherein the piston includes a rubber feature that seals against an interior of the housing.

14. A system for capturing unwanted materials within blood flowing through a patient's vasculature, the system comprising:
a blood filter adapted to capture the unwanted materials; and
the medical device of claim 8;
wherein the blood filter is adapted to be fluidly coupled with the fluid inlet; and
wherein blood exiting the fluid outlet is directed back to the patient.

15. The system of claim 14, wherein the blood exiting the fluid outlet is returned directly to the patient.

16. The system of claim 14, wherein the blood exiting the fluid outlet is captured and stored for subsequent return to the patient.

17. The system of claim 14, wherein the unwanted materials comprise embolic material.

18. The system of claim 14, wherein the unwanted materials comprise foreign matter not normally part of the patient's blood.

19. A medical device adapted for removing unwanted materials within blood flowing through a patient's vasculature, the medical device comprising:
a housing adapted to be held within a user's hand;
a pump secured within the housing, the pump including:
a cylinder formed within the housing;
a piston slidingly disposed within the cylinder;
a connecting rod pivotably secured to the piston; and
a rotating disk having a handle to rotate the disk, the connecting rod pivotably secured to the rotating disk at a position offset from a center of the rotating disk such that when the rotating disk is rotated, the connecting rod causes the piston to translate back and forth within the cylinder;
a fluid inlet fluidly coupled with the pump, the fluid inlet adapted to receive fluid pulled towards the pump; and
a fluid outlet fluidly coupled with the pump, the fluid outlet adapted to expel fluid away from the pump;
wherein as the pump is operated by the user, fluid is alternatively pulled into the fluid inlet and expelled through the fluid outlet.

20. The medical device of claim 19, further comprising a handle that is releasably securable relative to the rotating disk.
